(19) United States
(12) Patent Application Publication
(10) Pub. No.: US 2017/0196507 A1
(43) Pub. Date: Jul. 13, 2017

(54) SYSTEMS AND METHODS FOR MEASURING PERFORMANCE PARAMETERS RELATED TO ARTIFICIAL ORTHOPEDIC JOINTS

(71) Applicant: MiRus LLC, Atlanta, GA (US)

(72) Inventors: Angad Singh, Atlanta, GA (US); Philip Matthew Fitzsimons, Lilburn, GA (US)

(73) Assignee: MiRus LLC, Atlanta, GA (US)

(21) Appl. No.: 15/319,936

(22) PCT Filed: Jun. 19, 2015
(86) PCT No.: PCT/US2015/036775

§ 371 Date: Dec. 19, 2016

Related U.S. Application Data
(60) Provisional application No. 62/014,431, filed on Jun. 19, 2014.

(51) Int. Cl. A61B 5/00; A61B 5/11

(52) U.S. Cl. CPC A61B 5/4851; A61B 5/4528; A61B 5/4585; A61B 5/742; A61B 5/0031; A61B 5/1121; A61B 5/112; A61B 2562/0219; A61B 2562/0223

(57) ABSTRACT

A joint monitoring system for measuring performance parameters associated with an orthopedic articular joint comprises a force sensing module and an inertial measurement units. The sensing module comprises a housing that engages with the joint articular surface having a medial portion and a lateral portion. The sensing module also includes a first and second set of sensors disposed within the housing. The first set of sensors are mechanically coupled to the medial portion of the particular surface and configured to detect information of a force incident upon the medial portion of the articular surface. The second set of sensors are mechanically coupled to the lateral portion of the articular surface and configured to detect information a force incident upon a lateral portion of the articular surface. The inertial measurement unit is configured to detect an orientation of at least one of a first and second bone of a knee joint.

### CROSS-REFERENCE TO RELATED APPLICATION

This application claims the benefit of U.S. Provisional Application No. 62/014,431, filed Jun. 19, 2014, hereby incorporated herein by reference in its entirety.

### TECHNICAL FIELD

The present disclosure relates generally to artificial orthopedic joints and, more particularly, to systems and methods for measuring performance parameters associated with joint prosthetics.

### BACKGROUND

More than 800,000 total knee and hip replacements are performed in the US every year. This number is expected to increase to more than 4,000,000 by 2030. This trend of increasing joint replacements is the result of the improved quality of life that is typically the result of such procedures and the increasing acceptance of the procedure among the general population. Other reasons include an aging population with arthritis requiring joint replacement; the increasing prevalence of obesity, which puts undue stress on the knee and hip joints; the trend towards people remaining physically active later in life, which also places demands on the joints. The failure rate of joint replacements is between 10-20% over 10-20 years. Wear, loosening, mal-alignment, dislocation, and infection are typical causes of failure. Failures typically result in revision surgeries that are more technically challenging and correspondingly more risky than the original surgery. Therefore failures are devastating to the patient, frustrating for the surgeon, and costly to the healthcare system.

Given the above, there is a need to improve the performance and longevity of joint implants. Monitoring of post-operative joint performance parameters could enable early detection of potential issues providing the surgeon an opportunity to take preventative actions before the joint has deteriorated to point where major revision surgery is the only option. Such preventative actions could include non-invasive/minimally invasive interventions, physical therapy, medications and changes in patient lifestyle. Current methods for monitoring joint condition are imprecise and untimely since they mostly involve diagnosis based on pain, radiographic imaging, and physical examination without direct measurement of the biomechanics of the knee implant. Monitoring and trending of joint performance parameters such as the joint's load distribution, wear, and temperature could provide early indication of loosening, mal-alignment, and need for revision.

The presently disclosed systems and methods for post-operatively tracking joint performance parameters in orthopedic arthroplastic procedures are directed to overcoming one or more of the problems set forth above and/or other problems in the art.

### SUMMARY

According to one aspect, the present disclosure is directed to a computer-implemented method for tracking parameters associated with an orthopedic articular joint, the method comprising receiving, at a processor associated with a computer, first information indicative of a force detected at an articular interface between a first bone and a second bone of a patient and receiving, at the processor, second information indicative of an orientation of at least one of the first bone and the second bone. The method may further comprise estimating, by the processor, an orientation angle associated with at least one of the first bone and the second bone relative to a reference axis, the orientation angle, based, at least in part, on the second information. The method may further comprise receiving, at a processor associated with a computer, third information indicative of the wear of the joint bearing surface. The method may further comprise receiving, at a processor associated with a computer, fourth information indicative of the internal temperature of the joint.

In accordance with another aspect, the present disclosure is directed to an implantable sensing module for measuring performance parameters associated with an orthopedic articular joint. The sensing module includes a first set of force sensors, the first set of sensors being mechanically coupled to the medial portion of the articular surface and configured to detect information indicative of a first force incident upon the medial portion of the articular surface. The sensing module may also include a second set of force sensors, the second set of sensors being mechanically coupled to the lateral portion of the articular surface and configured to detect information indicative of a second force incident upon a lateral portion of the articular surface. The sensing module may further include one or more wear sensors configured to measure the wear of the joint bearing surface. The sensor module may also include a temperature sensor configured to measure the internal temperature of the joint which could be indicative of infection or other abnormal condition.

According to another aspect, the present disclosure is directed to a joint monitoring system for tracking performance parameters associated with an orthopedic articular joint that comprises an interface between a first bone and a second bone. The joint monitoring system comprises a sensing module configured for implantation within a prosthetic orthopedic articular joint. The sensing module may be configured to detect information indicative of at least one force incident upon at least a portion of an articular surface of the joint. The sensing module may further be configured to measure the wear of the bearing surface as well as the internal temperature of the joint. The sensing module may also comprise at least one inertial measurement unit for tracking the three-dimensional angles of the orthopedic articular joint. The joint monitoring system may further comprise a processing device, communicating with the sensing module. The processing device may be configured to estimate a location of at least one force relative to the articular surface, the estimated location based, at least in part, on the information indicative of the force incident upon at least a portion of the articular surface of the sensing module. The processing device may also be configured to estimate an orientation angle associated with at least one of the first bone and the second bone relative to a reference axis, the orientation angle, based, at least in part, on the information indicative of the orientation of at least one of the first bone and the second bone.

In accordance with another aspect, the present disclosure is directed to an implantable sensing module for measuring performance parameters associated with a prosthetic orthopedic articular joint. The sensing module may comprise a plurality of sensors disposed within a recess created on the tibial implant surface. The plurality of sensors may be mechanically coupled to the articular surface and configured to detect information indicative of a force incident upon the articular surface of the joint, an orientation of the implanted prosthesis, internal temperature of the joint, and/or wear of the bearing surface. The sensing module may also include a processing device, communicating with each of the plurality of sensors and configured to receive the above information. The processing device may also be configured to estimate a location of a center of the force relative to a boundary associated with the articular surface, and estimate a magnitude of the force at the estimated location of the center of the force.

### BRIEF DESCRIPTION OF THE DRAWINGS

FIG. 1 provides a diagrammatic view of an exemplary joint monitoring system.

FIG. 2 illustrates a magnified view of an exemplary reconstructed knee joint with a sensing module.

FIG. 3 provides a schematic view of exemplary components associated with a joint monitoring system, such as the joint monitoring system illustrated in FIG. 1.

FIG. 4 provides a perspective exploded view of an exemplary sensing module.

FIG. 5A provides a circuit diagram of an exemplary piezoelectric energy harvester.

FIG. 5B provides an alternative circuit diagram of an exemplary piezoelectric energy harvester.

FIG. 5C provides a circuit diagram of an exemplary radio frequency (RF) energy harvester.

FIG. 5D provides an alternative circuit diagram of an exemplary RF harvester.

FIG. 6A provides a schematic view of an exemplary sensing transducer shown in FIG. 6A.

FIG. 6B provides a schematic view of an exemplary capacitor-type force detecting transducer.

FIG. 6C provides a schematic view of another exemplary capacitor-type design of a force detecting transducer.

FIG. 7 illustrates an embodiment of a user interface that may be provided on a monitor or output device for displaying the monitored joint performance parameters in real time.

FIG. 8 provides an exemplary screenshot that displays the load magnitudes on the medial and lateral sides alongside the 3D joint angles throughout a patient's gait cycle.

FIG. 9 provides an exemplary screenshot of a trend that displays excessive load values on the medial side during walking and warns the surgeon via a visual, audible, or audiovisual alert.

FIG. 10 provides an exemplary screenshot of a trend that displays excessive wear values and warns the surgeon via a visual, audible, or audiovisual alert.

FIG. 11 provides a flowchart depicting an exemplary process associated with the user interface in FIG. 7 to be performed by one or more processing devices associated with monitoring systems.

### DETAILED DESCRIPTION

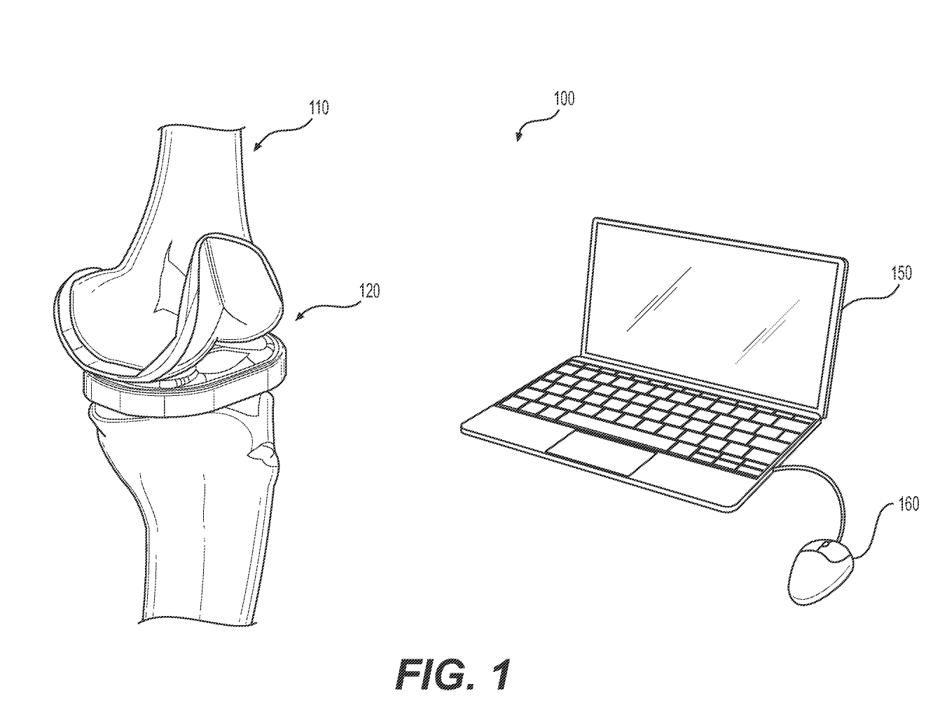

FIG. 1 provides a diagrammatic illustration of an exemplary joint monitoring system 100 for post-operative detection, monitoring, and tracking of performance parameters of an orthopedic joint, such as knee joint 120 of leg 110. For example, in accordance with the exemplary embodiment illustrated in FIG. 1, joint monitoring system 100 may embody a system for post-operatively gathering, analyzing, tracking, and trending performance parameters at knee joint 120 after a full or partial knee replacement procedure. Joint performance parameters may include or embody any parameter for characterizing the behavior or performance of an orthopedic joint. Non-limiting examples of joint performance parameters include any information indicative of force, pressure, temperature, wear of bearing surface, angle of flexion and/or extension, torque, varus/valgus displacement, location of center of force, axis of rotation, relative rotation of tibia and femur, tibial component rotation, range of motion, or orientation. Joint monitoring system 100 may be configured to monitor one or more of these exemplary performance parameters, track the parameters over time (and/or range of activities or motion), and display the monitored and/or tracked data to a surgeon or medical professional in real-time and/or as longitudinal trends. As such, joint monitoring system 100 provides a platform that facilitates post-operative evaluation of several joint performance parameters simultaneously.

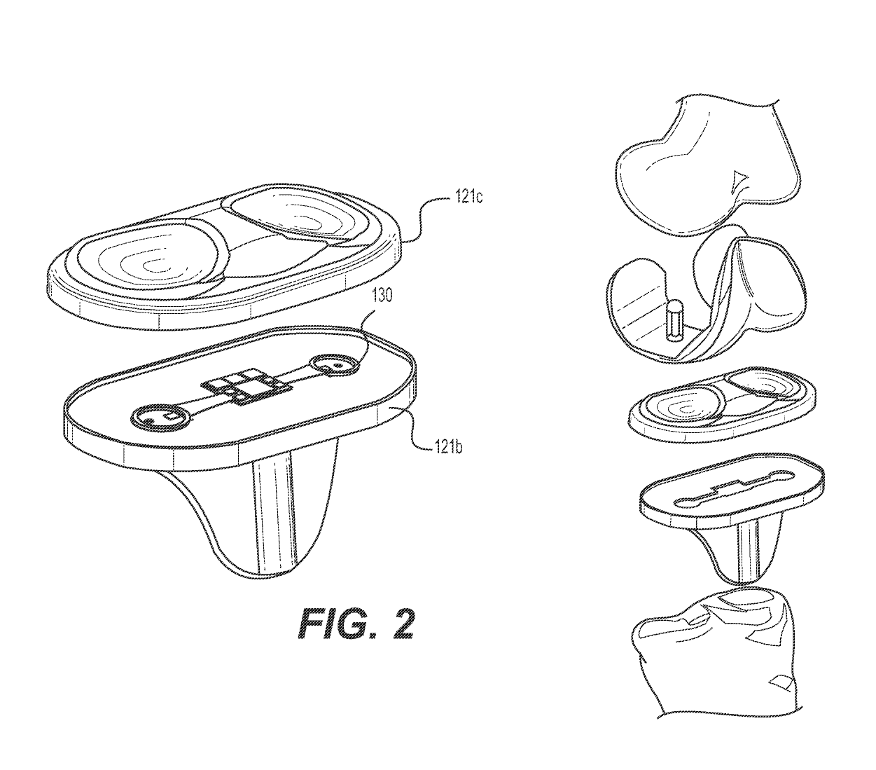

As illustrated in FIG. 1, joint monitoring system 100 may include a sensing module 130 (shown in FIG. 2), a processing device (such as processing system 150 (or other computer device for processing data received by sensing module 130)), and one or more wireless communication transceivers 160 for communicating with one or more of sensing module 130. The components of joint monitoring system 100 described above are exemplary only, and are not intended to be limiting. Indeed, it is contemplated that additional and/or different components may be included as part of joint monitoring system 100 without departing from the scope of the present disclosure. For example, although wireless communication transceiver 160 is illustrated as being a standalone device, it may be integrated within one or more other components, such as processing system 150. Thus, the configuration and arrangement of components of joint monitoring system 100 illustrated in FIG. 1 are intended to be exemplary only. Individual components of exemplary embodiments of joint monitoring system 100 will now be described in more detail.

Processing system 150 may include or embody any suitable microprocessor-based device configured to process and/or analyze information indicative of performance of the articular joint. According to one embodiment, processing system 150 may be a general purpose computer programmed for receiving, processing, and displaying information indicative of kinematic and/or kinetic parameters associated with the articular joint. According to other embodiments, processing system 150 may be a special-purpose computer, specifically designed to communicate with, and process information for, other components associated with joint monitoring system 100. Individual components of, and processes/methods performed by, processing system 150 will be discussed in more detail below.

Processing system 150 may communicate with one or more of sensing module 130 and configured to receive, process, and/or analyze data monitored by sensing module 130. According to one embodiment, processing system 150 may be wirelessly coupled to sensing module 130 via wireless communication transceiver(s) 160 operating any suitable protocol for supporting wireless (e.g., wireless USB, ZigBee, Bluetooth, Wi-Fi, etc.) In accordance with another embodiment, processing system 150 may be wirelessly coupled to sensing module 130, which, in turn, may be configured to collect data from the other constituent sensors and deliver it to processing system 150.

Wireless communication transceiver(s) 160 may include any suitable device for supporting wireless communication between one or more components of joint monitoring system 100. As explained above, wireless communication transceiver(s) 160 may be configured for operation according to any number of suitable protocols for supporting wireless, such as, for example, wireless USB, ZigBee, Bluetooth, Wi-Fi, or any other suitable wireless communication protocol or standard. According to one embodiment, wireless communication transceiver 160 may embody a standalone communication module, separate from processing system 150. As such, wireless communication transceiver 160 may be electrically coupled to processing system 150 via USB or other data communication link and configured to deliver data received therein to processing system 150 for further processing/analysis. According to other embodiments, wireless communication transceiver 160 may embody an integrated wireless transceiver chipset, such as the Bluetooth, Wi-Fi, NFC, or 802.11x wireless chipset included as part of processing system 150.

Sensing module 130 may include a plurality of components that are collectively adapted for implantation within at least a portion of an articular joint and configured to detect various static and dynamic parameters present at, on, and/or within the articular joint. According to one embodiment (and as shown in FIG. 1), sensing module 130 may embody a tibial implant prosthetic component configured for insertion within a fully reconstructed knee joint 120. As shown in FIG. 2, the bottom surface of the sensing module 130 is configured to engage with tibial prosthetic component 121b attached to a resected portion of the patient's tibia while the top surface is configured to engage with polyethylene insert 121c or any other material designed to act as bearing surface of the implant. For example, according to one embodiment, sensing module 130 may be configured for insertion and coupling to a top surface of a plate positioned atop a prosthetic component designed to replace a resection portion of a patient's tibia. A top surface of sensing module 130 may be adapted to receive an insert that is configured to serve as the load bearing surface that is designed to interact with the prosthetic femoral component of the reconstructed joint. Once knee joint 120 is reconstructed, sensing module 130 may be configured to detect various performance parameters at knee joint 120 post-operatively. Exemplary components and subsystems associated with sensing module 130 will be described in more detail below.

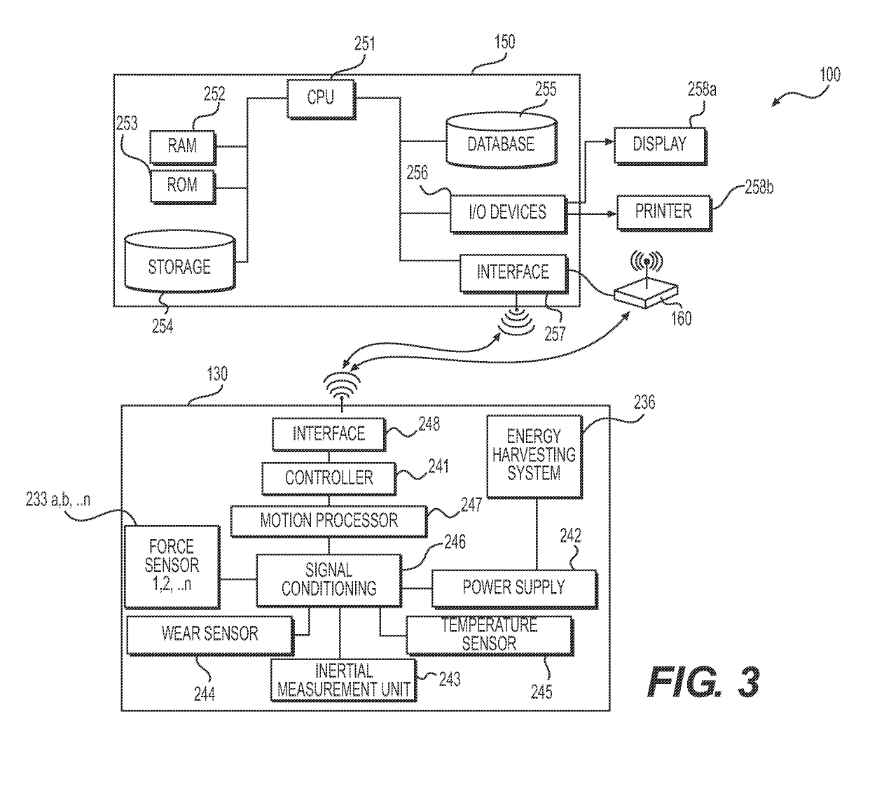

Sensing module 130 may include inertial measurement unit(s) 243 (shown in FIG. 3) that may be any system suitable for measuring information that can be used to accurately measure orientation in one or more spatial dimensions. From this orientation information the joint angles such as flexion and/or extension of the orthopedic joint can be derived. Joint flexion (and/or extension) data can be particularly useful in evaluating the stability of the joint as the leg is flexed and extended. Inertial measurement units have their own reference coordinate frames and report their orientation with respect to that frame. Inertial measurement unit 243 is configured to measure the relative orientation of a bone with respect to a reference orientation, such as the orientation of the respective sensor when the leg is positioned in a fully extended pose (0 degrees flexion) with no internal/external rotation or varus/valgus forces applied. It should be noted that although in the exemplary embodiment as shown in FIG. 3, the inertial measurement unit 243 is embedded in sensing module 130, inertial measurement unit 243 can be attached to any feature of the patient's anatomy that will provide information indicative of the flexion (and/or extension) of the joint and may be worn as an external unit separate from the implant.

FIG. 2 provides a magnified view of knee joint 120 showing sensing module 130 coupled to tibial component 121b and configured to engage with polyethylene insert 121c. In this embodiment, sensing module 130 is embedded in tibial implant component 121b that is permanently implanted in the knee joint 120. As shown in FIG. 2, sensing module 130 may be adapted for insertion into a corresponding tray feature associated with tibial component 121b. According to one embodiment, sensing module may include a piezoelectric energy harvesting stack that is disposed in a column that extends from the underside of the sensing module 130. This column is configured for insertion into a corresponding well disposed in the surface of the tray feature associated with tibial component 121b. In addition to providing an efficient housing for the energy harvesting stack, this column feature (and corresponding well) aids in maximizing the load experienced by the piezoelectric stack (and hence the power harvested) as well as maintaining stable alignment and position of the sensing module 130.

FIG. 3 provides a schematic diagram illustrating certain exemplary subsystems associated with joint monitoring system 100 and its constituent components. Specifically, FIG. 3 is a schematic block diagram depicting exemplary subcomponents of processing system 150 and sensing module 130, in accordance with certain disclosed embodiments.

As explained, processing system 150 may be any processor-based computing system that is configured to receive kinematic and/or kinetic parameters associated with an orthopedic joint 120, analyze the received parameters to extract data indicative of the performance of orthopedic joint 120, and output the extracted data in real-time or near real-time. Non-limiting examples of processing system 150 include a desktop or notebook computer, a tablet device, a smartphone, a wearable computer or any other suitable processor-based computing system. Furthermore, as explained previously, processing system 150 is a networked computer and certain memory components (e.g., database 255) associated with processing system 150 may be, in whole or in part, implemented as a distributed memory system, such as a cloud-based memory store, or a multi-device network-based storage device.

For example, as illustrated in FIG. 3, processing system 150 may include one or more hardware and/or software components configured to execute software programs, such as software tracking kinematic and/or kinetic parameters associated with orthopedic joint 120 and displaying information indicative of the kinematic and/or kinetic performance of the joint. According to one embodiment, processing system 150 may include one or more hardware components such as, for example, a central processing unit (CPU) 251, a random access memory (RAM) module 252, a read-only memory (ROM) module 253, a memory or data storage module 254, a database 255, one or more input/output (I/O) devices 256, and an interface 257. Alternatively and/or additionally, processing system 150 may include one or more software media components such as, for example, a computer-readable medium including computer-executable instructions for performing methods consistent with certain disclosed embodiments. It is contemplated that one or more of the hardware components listed above may be implemented using software. For example, storage 254 may include a software partition associated with one or more other hardware components of system 150. Processing system 150 may include additional, fewer, and/or different components than those listed above. It is understood that the components listed above are exemplary only and not intended to be limiting.

CPU 251 may include one or more processors, each configured to execute instructions and process data to perform one or more functions associated with processing system 150. As illustrated in FIG. 3, CPU 251 may communicate with RAM 252, ROM 253, storage 254, database 255, I/O devices 256, and interface 257. CPU 251 may be configured to execute sequences of computer program instructions to perform various processes, which will be described in detail below. The computer program instructions may be loaded into RAM 252 for execution by CPU 251.

RAM 252 and ROM 253 may each include one or more devices for storing information associated with an operation of processing system 150 and/or CPU 251. For example, ROM 253 may include a memory device configured to access information associated with processing system 150, including information for identifying, initializing, and monitoring the operation of one or more components and subsystems of processing system 150. RAM 252 may include a memory device for storing data associated with one or more operations of CPU 251. For example, ROM 253 may load instructions into RAM 252 for execution by CPU 251.

Storage 254 may include any type of mass storage device configured to store information that CPU 251 may need to perform processes consistent with the disclosed embodiments. For example, storage 254 may include one or more magnetic and/or optical disk devices, such as hard drives, CD-ROMs, DVD-ROMs, or any other type of mass media device. Alternatively or additionally, storage 254 may include flash memory mass media storage or other semiconductor-based storage medium.

Database 255 may include one or more software and/or hardware components that cooperate to store, organize, sort, filter, and/or arrange data used by processing system 150 and/or CPU 251. For example, database 255 may include historical data such as, for example, stored kinematic and/or kinetic performance data associated with the orthopedic joint. CPU 251 may access the information stored in database 255 to provide a performance comparison between previous joint performance and current (i.e., real-time) performance data. CPU 251 may also analyze current and previous kinematic and/or kinetic parameters to identify trends in historical data (i.e., the forces detected at medial and lateral articular surfaces at various post-operative intervals for one or more patient activities). These trends may then be recorded and analyzed to allow the surgeon or other medical professional to compare the data at various stages of the knee replacement procedure. It is contemplated that database 255 may store additional and/or different information than that listed above. Database 255 may also be implemented as virtual database on the “cloud” which can be accessed by processing system 150 via the internet. The database 255 may also be accessed remotely by physicians using internet connected computers and/or hand-held devices

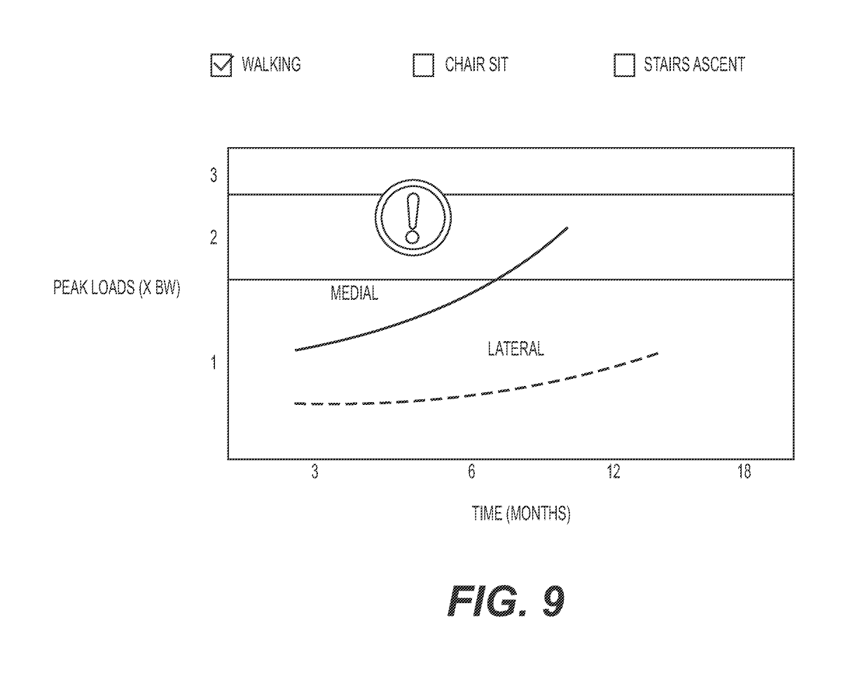

I/O devices 256 may include one or more components configured to communicate information with a user associated with joint monitoring system 100. For example, I/O devices may include a console with an integrated keyboard and mouse to allow a user to input parameters associated with processing system 150. I/O devices may also include a microphone for voice commands or a camera for gesture-based commands. Other gesture-based technologies such as those utilizing motion sensors may also be utilized. I/O devices 256 may also include a display including a graphical user interface (GUI) (such as GUI 900 shown in FIG. 9) for outputting information on a display monitor 258a. I/O devices 256 may also include peripheral devices such as, for example, a printer 258b for printing information associated with processing system 150, a user-accessible disk drive (e.g., a USB port, a floppy, CD-ROM, or DVD-ROM drive, etc.) to allow a user to input data stored on a portable media device, a microphone, a speaker system, or any other suitable type of interface device.

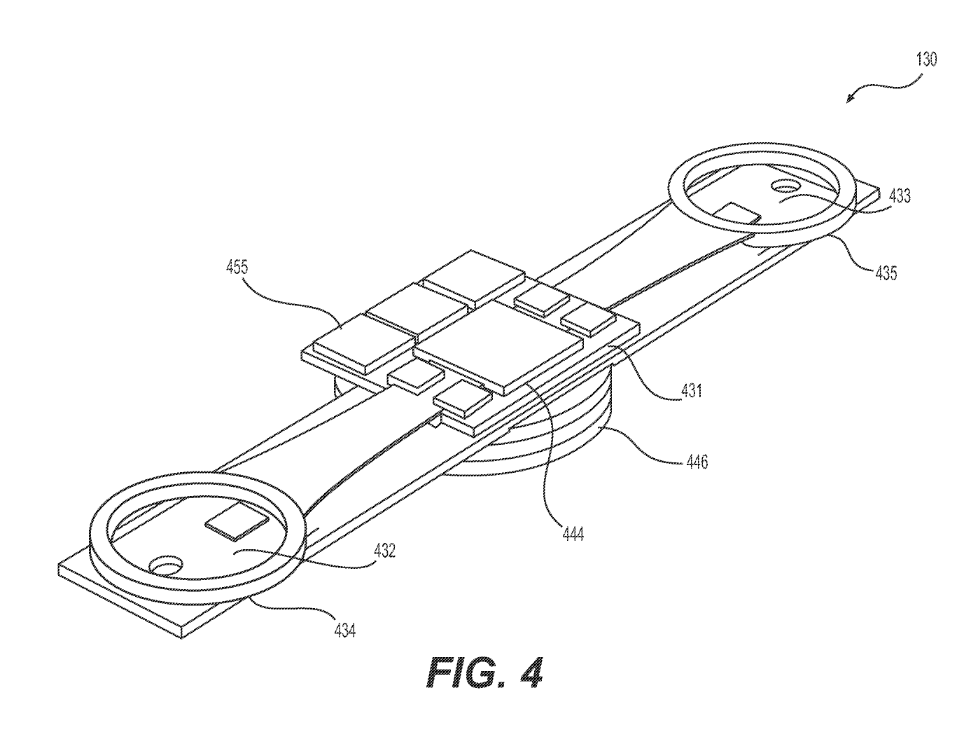

Interface 257 may include one or more components configured to transmit and receive data via a communication network, such as the Internet, a local area network, a workstation peer-to-peer network, a direct link network, a wireless network, or any other suitable communication platform. For example, interface 257 may include one or more modulators, demodulators, multiplexers, demultiplexers, network communication devices, wireless devices, antennas, modems, and any other type of device configured to enable data communication via a communication network. According to one embodiment, interface 257 may be coupled to or include wireless communication devices, such as a module or modules configured to transmit information wirelessly using Wi-Fi or Bluetooth wireless protocols. Alternatively or additionally, interface 257 may be configured for coupling to one or more peripheral communication devices, such as wireless communication transceiver 160. Sensing module 130 may include a plurality of subcomponents that cooperate to detect one or more of force, temperature, wear, and/or joint orientation information at orthopedic joint 120, and transmit the detected data to processing system 150 for further analysis. According to one exemplary embodiment, sensing module 130 may include a controller 241, a power supply 242, an energy harvesting system 236, an interface 248, and one or more force sensors 233a, 233b, . . . 233n, wear sensors 244, temperature sensors 245, and inertial measurement unit 243 coupled to signal conditioning circuits 246. Those skilled in the art will recognize that the listing of components of sensing module 130 is exemplary only and not intended to be limiting. Indeed, it is contemplated that sensing module 130 may include additional and/or different components than those shown in FIG. 3. For example, although FIG. 3 illustrates controller 241, signal conditioning 246, and interface 248 as separate components, it is contemplated that these components may embody one or more modules (either distributed or integrated) within a single microprocessor. Exemplary subcomponents of sensing module 130 will be described in greater detail below with respect to FIG. 4.

As explained, sensing module 130 may contain a inertial measurement unit 243 that may include one or more subcomponents configured to detect and transmit information that either represents a three-dimensional orientation or can be used to derive an orientation of the inertial measurement unit 243 (and, by extension, any object rigidly affixed to inertial measurement unit 243, such as a tibia and femur of a patient). Inertial measurement unit 243 may embody a device capable of determining a three-dimensional orientation associated with any body to which inertial measurement unit 243 is attached. According to one embodiment, inertial measurement unit 243 may include one or more of a gyroscope, one or more of an accelerometer, or one or more of a magnetometer.

Fewer of these devices can be used without departing from the scope of the present disclosure. For example, according to one embodiment, inertial measurement units may include only a gyroscope and an accelerometer, the gyroscope for calculating the orientation based on the rate of rotation of the device, and the accelerometer for measuring earth's gravity and linear motion, the accelerometer providing corrections to the rate of rotation information (based on errors introduced into the gyroscope because of device movements that are not rotational or errors due to biases and drifts). In other words, the accelerometer may be used to correct the orientation information collected by the gyroscope. Similar a magnetometer can be utilized to measure the earth's magnetic field and can be utilized to further correct gyroscope errors. Thus, while all three of gyroscope, accelerometer, and magnetometer may be used, orientation measurements may be obtained using as few as one of these devices. The use of additional devices increases the resolution and accuracy of the orientation information and, therefore, may be preferable in embodiments where resolution is critical.

Controller 241 may be configured to control and receive conditioned and processed data from one or more of force sensors 233, wear sensor 244, temperature sensor 245, and inertial measurement unit 243 and transmit the received data to one or more remote receivers. The data may be pre-conditioned via signal conditioning circuitry 246 consisting of amplifiers and analog-to-digital converters or any such circuits. The signals may be further processed by a motion processor 247. Motion processor 247 may be programmed with “motion fusion” algorithms to collect and process data from different sensors to generate error corrected orientation information. Accordingly, controller 241 may communicate (e.g., wirelessly via interface 248 as shown in FIG. 3, or using a wireline protocol) with, for example, processing system 150 and configured to transmit the data received from one or more sensors processing system 150, for further analysis. Interface 248 may include one or more components configured to transmit and receive data via a communication network, such as the Internet, a local area network, a workstation peer-to-peer network, a direct link network, a wireless network, or any other suitable communication platform. For example, interface 248 may include one or more modulators, demodulators, multiplexers, demultiplexers, network communication devices, wireless devices, antennas, modems, and any other type of device configured to enable data communication via a communication network. According to one embodiment, interface 248 may be coupled to or include wireless communication devices, such as a module or modules configured to transmit information wirelessly using Wi-Fi or Bluetooth wireless protocols.

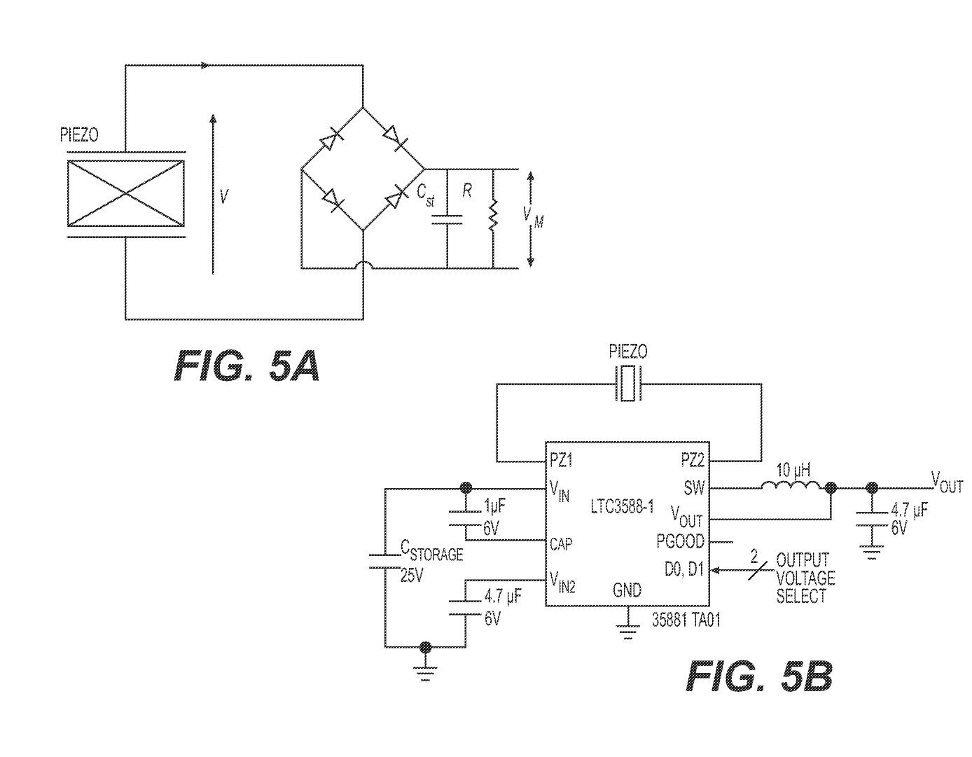

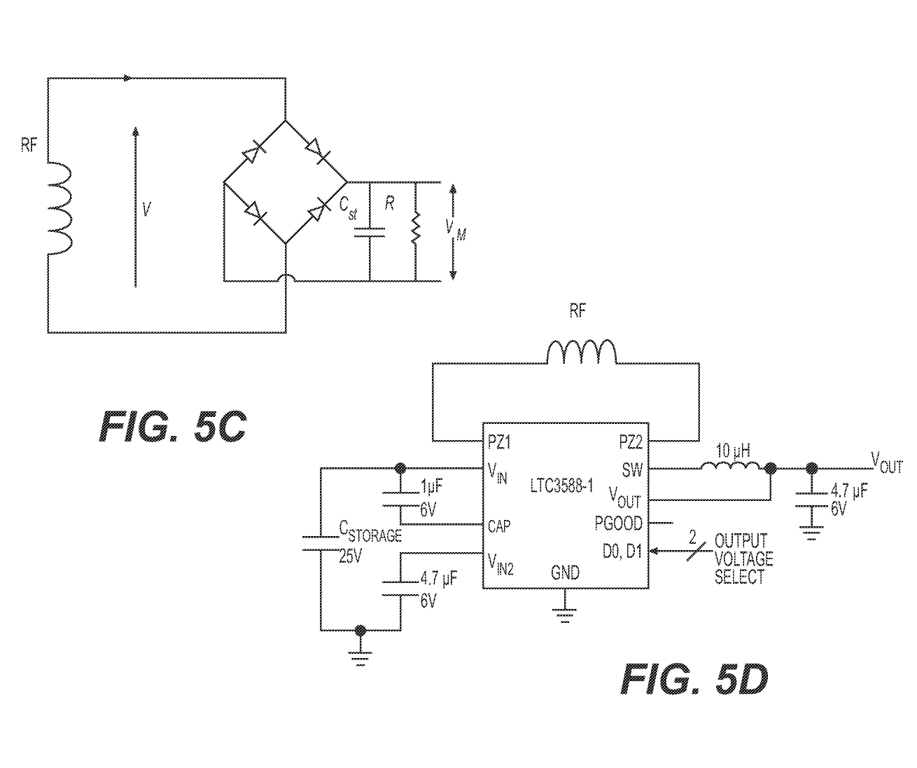

As illustrated in FIG. 3, sensing module 130 may be powered by power supply 242, such as a battery, fuel cell, MEMs micro-generator, or any other suitable compact power supply. Power supply 242 may be a rechargeable battery or power storage device that can charged wirelessly via inductive coupling, transmitted RF energy, ultrasound or other such wireless power transfer techniques know in the art. Alternatively or in combination with the above, a suitable energy harvesting system 236 may be implemented. Any suitable energy harvesting system such as those based on piezoelectric, radio frequency (RF), or thermal may be implemented. Since during normal course of patient activity, the knee joint is subjected to high forces, piezoelectric energy harvesting is particularly attractive. A piezoelectric energy harvesting system converts mechanical strain energy into electrical energy. Enough energy may be harvested to power the sensing module 130 periodically or continuously. A piezoelectric energy harvesting system suitable for use in sensing module 130 may consist of a piezoelectric transducer stack 446 and associated signal conditioning and energy storage circuitry. An example of a commercially available piezoelectric stack that can be used is the TS18-H5-104 from Piezo Systems, Woburn, Mass. To maximize the load experienced by the stack and therefore the energy harvested the stack is placed in optimal alignment to the direction of the load and module 130 is mechanically designed so that a significant portion of the load experienced by the joint may be transferred to the stack. The output voltage of piezoelectric stack is typically rectified and then used to store energy in a storage capacitor such as a super capacitor. Such a basic circuit for energy harvesting is shown in FIG. 5A. For more optimal energy harvesting a buck converter may be included. Piezoelectric energy harvesting circuits are now commercially available and may be incorporated in the invention. An example of such a commercially available solution is the LTC3588-1 from Linear Technologies. An example of circuit utilizing the LTC3588-1 is shown in FIG. 5B. FIGS. 5C and 5D show alternative embodiments of the energy harvesting circuits for harvesting RF energy.

FIG. 4 illustrates an exploded perspective view of sensing module 130, consistent with certain disclosed embodiments. Sensing module 130 may include an electronic circuit board 431, such as printed circuit board (PCB), multi-chip module (MCM), or flex circuit board, configured to provide both integrated, space-efficient electronic packaging and mechanical support for the various electrical components and subsystems of sensing module 130. Sensing module 130 may also include controller 241 and interface 248 (shown as microcontroller system-on-chip with integrated RF transceiver 444 in FIG. 4), a first force sensor 432 associated with medial portion of sensing module 130, a second force sensor 433 associated with lateral portion of sensing module 130, a power supply (not shown in FIG. 4, but shown as power supply 242 of FIG. 3), signal conditioning circuitry 246, and (optionally) one or more inertial measurement units 445 for detecting the orientation of sensing module 130 relative to a reference position. In addition to power supply 242, an energy harvesting system (partially shown as 446 in FIG. 4, but shown as energy harvesting system 236 of FIG. 3) may be implemented as a primary energy source or to supplement power supply 232. Energy harvesting system 236 may include or embody any suitable device (such as piezo stack 446) for generating or harvesting energy during normal operation of the device, and storing the harvested energy (using a capacitor, battery, or other charge storage device) or using the harvested energy to power the device.

Microcontroller 444 (and/or controller 241 and interface 248) may be configured to receive data from one or more of force sensors 432, 433, one or more wear sensors 434, 435, one or more temperature sensors (not shown in FIG. 4 but 245 in FIG. 3), and inertial measurement unit 445, and transmit the received data to one or more remote receivers. The data may be pre-conditioned via signal conditioning circuitry 246 consisting of amplifiers and analog-to-digital converters or any such circuits. The signal conditioning circuitry may also be used to condition the power supply voltage levels to provide a stable reference voltage for operation of the sensors. Accordingly, microcontroller 444 may include (or otherwise be coupled to) an interface 248 that may consist of a wireless transceiver chipset with or without an external antenna, and may be configured to communicate (e.g., wirelessly as shown in FIG. 3, or using a wireline protocol) with, for example, processing system 150. As such, microcontroller 444 may be configured to transmit the data received from one or more of sensors to processing system 150, for further analysis. Interface 248 may include one or more components configured to transmit and receive data via a communication network, such as the Internet, a local area network, a workstation peer-to-peer network, a direct link network, a wireless network, or any other suitable communication platform. For example, interface 248 may include one or more modulators, demodulators, multiplexers, demultiplexers, network communication devices, wireless devices, antennas, modems, and any other type of device configured to enable data communication via a communication network. According to one embodiment, interface 248 may be coupled to or include wireless communication devices, such as a module or modules configured to transmit information wirelessly using Wi-Fi or Bluetooth wireless protocols.

Sensing module 130 may optionally include an inertial measurement unit 445 to provide orientation (and/or position) information associated with sensing module 130 relative to a reference orientation (and/or position). Inertial measurement unit 445 may include one or more subcomponents configured to detect and transmit information that either represents an orientation or can be used to derive an orientation of the inertial measurement unit 445 (and, by extension, any object that is rigidly affixed to inertial measurement unit 445, such as a tibial component which is further attached to the tibia of the patient). Inertial measurement unit 445 may embody a device capable of determining a three-dimensional orientation associated with any body to which inertial measurement unit 445 is attached. According to one embodiment, inertial measurement unit 445 may include one or more of a gyroscope, an accelerometer, or a magnetometer.

As illustrated in FIGS. 3 and 4, sensing module 130 may include a plurality of force sensors, each configured to measure respective force acting on the sensor. The type and number of force sensors provided within sensing module 130 can vary depending upon the resolution and the desired amount of data. For example, one sensor could be used if the design goal of sensing system 130 is to simply detect the magnitude of force present at the tibiofemoral interface. If, however, the design goal of sensing system 130 is to not only provide the magnitude of the forces present at the tibiofemoral interface, but also estimate the distribution of the applied force, then additional sensors (as few as two) should be used to provide a sufficient number of data points.

As illustrated in FIG. 4, sensing module 130 may include a first force sensor 432 and a second force sensor 433. According to one embodiment, the first sensor 432 may be mechanically coupled to the underside of medial portion of polyethylene insert 121c. Similarly, the second sensor 433 may be mechanically coupled to the underside of lateral portion of polyethylene insert 121c. As such, the first force sensor 432 may be configured to detect forces incident on medial portion of the knee implant, while the second force sensor 433 may be configured to detect forces incident on lateral portions of the knee implant.

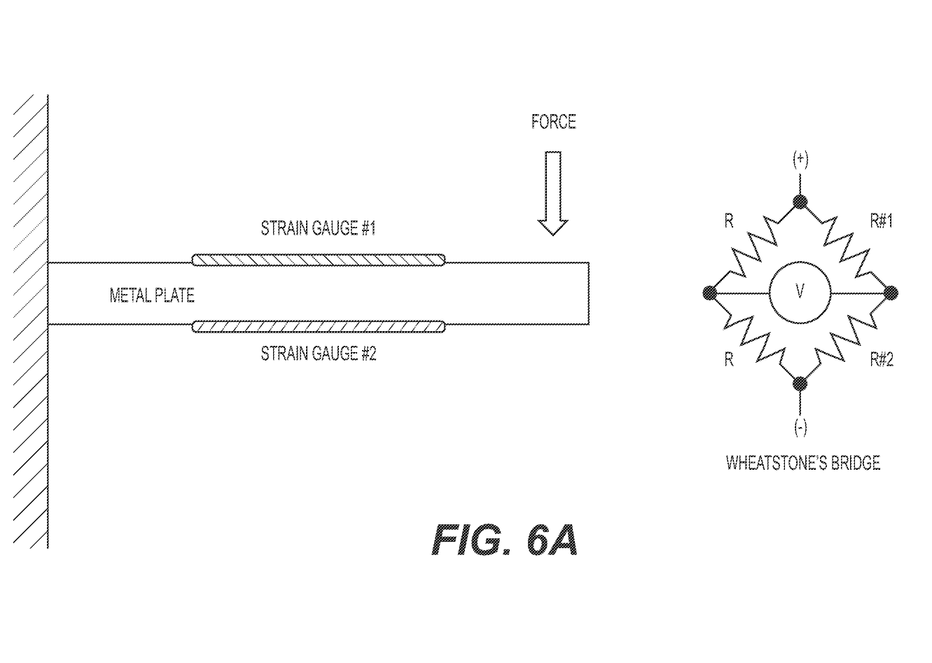

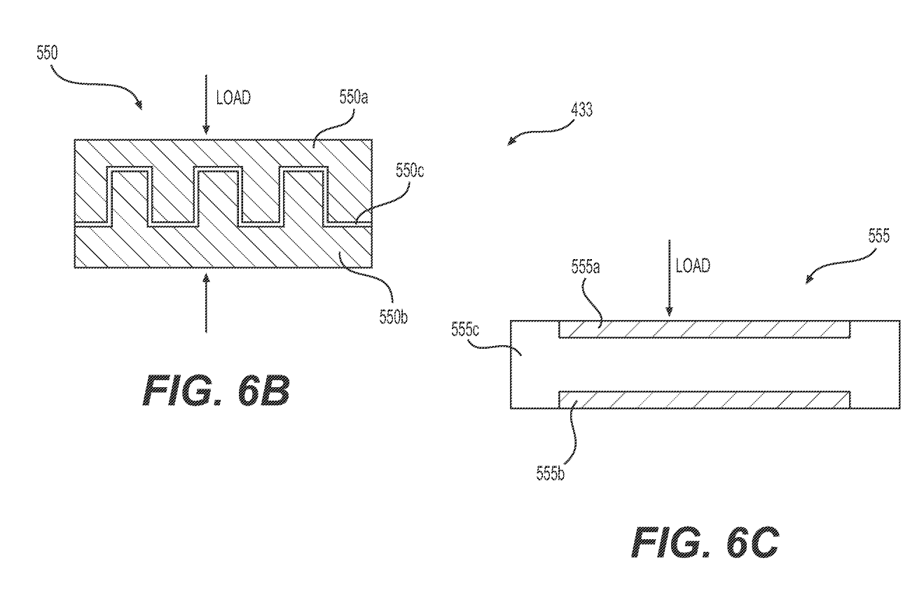

Force sensors 432 and 433 may be configured using a variety of different resistive or capacitive strain gauges for detecting applied force and/or pressure. Force sensors 432 and 433 each comprise two primary components: a metric portion that has a prescribed mechanical force-to-deflection characteristic and a measuring portion for accurately measuring the deflection of the metric portion and converting this measurement into an electrical output signal (using, for example, strain gauges). FIG. 6A illustrates a design for the metric and measuring portion of the force sensor in an exemplary embodiment of the invention.

Specifically, FIG. 6A illustrates a cantilever design with a prescribed force-to-deflection characteristic. Although certain embodiments are described as having force sensors that are cantilever-type, it is contemplated that force sensors may be based on other mechanical deformation principles, and any of which may be used in different exemplary embodiments. For example, force sensors 432 and 433 may embody at least one type of the following configurations of force sensors: binocular, ring, shear, or direct stress or spring torsion (including helical, disc, etc.) The measuring portion used with the above configurations may comprise strain gauges that can be either resistive, piezoresistive, capacitive, optical, magnetic or any such transducers that convert a mechanical deflection and/or strain to a measurable electrical parameter. Alternatively or additionally, any suitable resistive strain gauge, whose output resistance value changes with respect to the application of mechanical force, can be used as force sensors 432, 433. In certain embodiments, the resistive strain gauge could be the transducer class S182K series strain gauges from Vishay Precision Group, Wendell N.C.

Because the structures used in resistive sensors tend to exhibit relatively small changes in resistance under mechanical stress, a separate electrical circuit that is capable of detecting such small changes may be required. According to one embodiment, a Wheatstone bridge circuit may be used to measure the static or dynamic electrical resistance due to small changes in resistance due to mechanical strain.

As an alternative or in addition to resistive strain gauges, force sensors 432 and 433 may embody capacitive-type strain gauges. Capacitive-type strain gauges, such as those illustrated in the embodiments shown in FIGS. 6B and 6C, typically comprise two metal conductors fashioned as layers or plates separated by a dielectric layer. The dielectric layer may comprise a compressible material, such that when force is applied to one or more of the metal plates the dielectric layer compresses and changes the distance between the metal plates. This change in distance causes a change in the capacitance, which can be electrically measured and converted into a force value.

Exemplary designs of capacitive-type force sensors are illustrated in FIGS. 6B and 6C. For example, FIG. 6B illustrates capacitive-type sensor 550 with a lateral comb configuration (i.e., having a serpentine dielectric channel 550c separating metal plates 550a and 550b). Because this lateral-comb configuration effectively comprises several capacitors (at each of the interlocking comb-teeth), a lateral comb capacitive sensor 550 functions across a relatively large range of forces and exhibits good sensitivity and signal to noise ratio.

According to another exemplary embodiment shown in FIG. 6C, capacitive-type force sensor may embody a more conventional parallel-plate capacitor device 555, with metal plates 555a and 555b arranged in parallel around a dielectric layer 555c. Although less sensitive to compressive forces, parallel plate designs are simpler and less expensive to implement, and can be fairly accurate over smaller ranges of compressive forces.

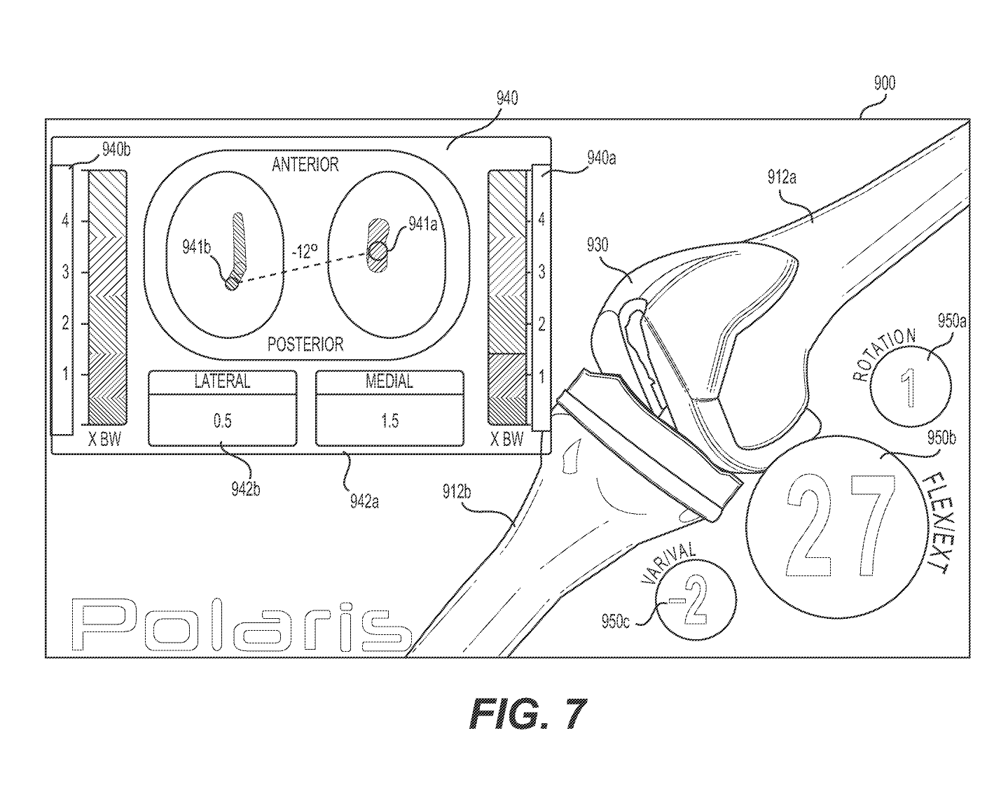

Processes and methods consistent with the disclosed embodiments provide a system for monitoring multiple parameters present at an orthopedic joint 120 and the three-dimensional alignment and/or angles of the joint, and can be particularly useful in post-operatively evaluating the performance of the joint. As explained, while various components, such as sensing module 130 can monitor various physical parameters (e.g., magnitude and location of force, wear, temperature, orientation, etc.) associated with the bones and interfaces that make up orthopedic joint 120, processing system 150 provides a centralized platform for collecting and compiling the various physical parameters monitored by the individual sensing units of the system, analyzing the collected data, and presenting the collected data in a meaningful way. FIGS. 7, 8, 9, and 10 illustrate exemplary processes and features associated with how processing system 150 performs the data analysis and presentation functions associated with sensing system 100.

FIG. 7 provides an exemplary screen shot 900 corresponding to a graphical user interface (GUI) associated with processing system 150. Screen shot 900 may correspond to embodiments in which sensing module 130 is configured to detect forces present at orthopedic joint 120. Specific details for each of this screen shots will be described in detail below with respect to the exemplary processes and methods performed by processing system 150, as outlined in FIG. 11.

As illustrated in FIG. 11, the process may commence when processing system 150 receives force measurement information from sensing module 130 (Step 1002) and/or orientation information from sensing module 130 (Step 1004). As explained, processing system 150 may include one or more communication modules for wirelessly communicating data with sensing module 130. As such, processing system 150 may be configured establish a continuous communication channel with sensing module 130 and automatically receive kinematic and/or kinetic data across the channel. Alternatively or additionally, processing system 150 may send periodic requests to sensing module 130 and receive updated kinematic and/or kinetic parameters in response to the requests. In either case, processing system 150 receives force and orientation information in real-time or near real-time.

Processing system 150 may be configured to determine a magnitude and/or location of the force detected by sensing module 130 (Step 1012). In certain embodiments, sensing module 130 may be configured to determine the location of the force relative to the boundaries of the articular surface. In such embodiments, processing system 150 may not necessarily need to determine the location, since the determination was made by sensing module 130.

In other embodiments, processing system 150 simply receives raw force information (i.e., a point-force value) from each sensor of sensing module 130, along with data identifying which force sensor detected the particular force information. In such embodiments, processing system 150 may be configured to determine the location of the force, by based on the relative value of a magnitude and the position of the force sensor within the sensing module 130.

Processing system 150 may also be configured to determine an angle of flexion/extension of joint 120 based on the orientation information received from inertial measurement unit(s) 243 (Step 1014). For example, processing system 150 may be configured to receive pre-processed and error-corrected orientation information from the inertial measurement unit(s) 243. Alternatively, processing system 150 may be configured to receive raw data from one or more of gyroscope, accelerometer, and/or magnetometer and derive the orientation based on the received information using known processes for determining orientation based on rotation rate data from gyroscope, acceleration information from accelerometer, and magnetic field information from magnetometer. In order to enhance precision of the orientation information, data from multiple units may be used to correct data from any one of the units. For example, accelerometer and/or magnetometer data may be used to correct error in rotation rate information due to gyroscope bias and drift issues. Optional temperature sensor information may also be utilized to correct for temperature effects.

Once processing system 150 has determined the magnitude and location of the force detected by the force sensors and joint angles, processing system 150 may analyze and compile the data for presentation in various formats that may be useful to a user of sensing system 100 (Step 1022). For example, as shown in FIG. 7, processing system 150 may be configured to display the instantaneous magnitude and location of the force (display area 940) on a portion of the GUI 900. According to one embodiment, software associated with processing system 150 may provide graphs 940a, 940b indicating the relative magnitude of the force detected at the respective sensors associated with medial and lateral portions of the prosthetic joint. As can be seen in FIG. 7, graphs 940a, 940b may include vertical gauges indicating the various force values that are detectable by processing system 150, along with a horizontal line that shows the instantaneous magnitude of the force value with respect to the gauge of possible values. As an alternative or in addition to graphs 940a, 940b, processing system 150 may be configured to simply display the numerical value of the medial and lateral forces (in any suitable unit of measurement such as times body weight), as shown in user interface elements 942a, 942b.

In addition to magnitude values, processing system 150 may include a user interface element configured to display the instantaneous locations 941a, 941b of the medial and lateral forces relative to the boundaries of the articular surface. In addition to the location, the graphical element may also be configured to adjust the size of the cursor or icon used to convey the location information to indicate the relative magnitude of the force value. In addition, certain previously-measured data (such as the location data) may be tracked and overlaid in the medial and lateral portions of the user interface, to provide the user with a view of the amount by which the location of the center of the load changes as the joint is flexed and extended. It should be noted that various other information can be provided as a user interface element associated with GUI 900.

For example, as an alternative or in addition to the magnitude and force presentation described above with respect to user interface region 940, processing system 150 may include user interface elements 950a, 950b, 950c that provides information indicative of the instantaneous values for flexion/extension (950b), internal/external rotation (950a), and varus/valgus alignment (950c), each of which processing system 150 can determine based on the three-dimensional orientation information from inertial measurement unit 243 (Step 1024). As part of this display element, processing system may also display graphical representations of femur 912a, tibia 912b, and implant 930, based on the instantaneous position data received from inertial measurement unit 243. The graphical representation may consist of an artificial model of the knee representing an approximation of the patient's knee, animated in real-time as the joint angles change in response to articulation of the joint by the surgeon. Alternatively, in the case where 3D image of the patient's joint is available, an anatomically correct 3D model of the patient's knee may be created by the processing unit 150 and animated in real-time.

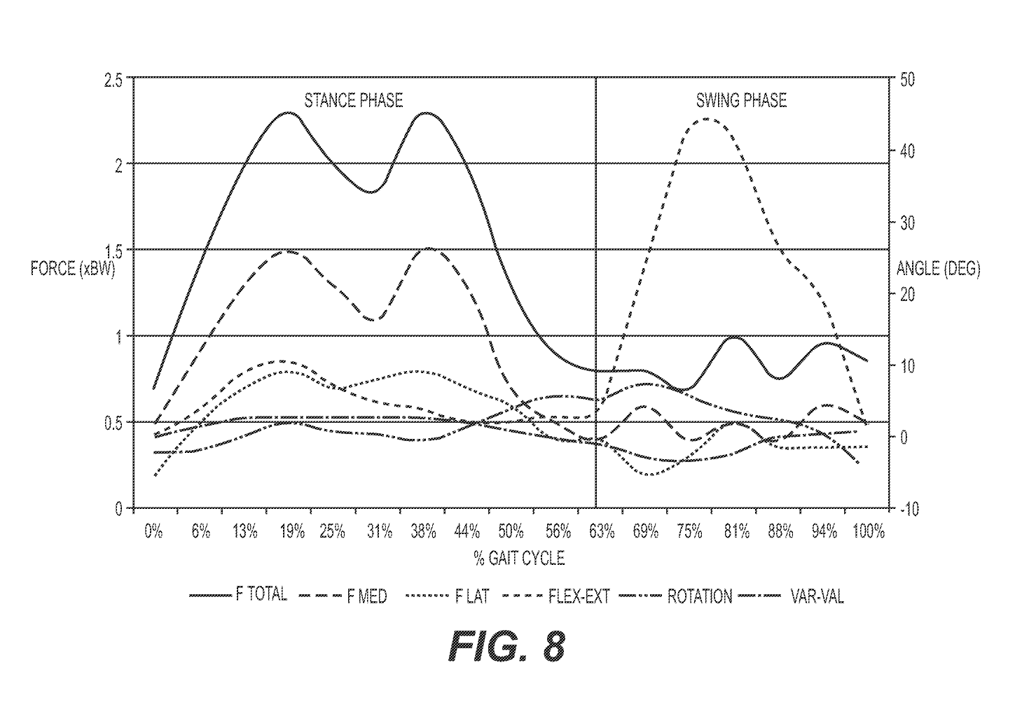

Alternatively, FIG. 8 provides an exemplary screenshot that displays the load magnitudes on the medial and lateral sides alongside the 3D joint angles throughout a patient's gait cycle. Such a view can be utilized as for an assessment of gait biomechanics. The processing system 150 may also incorporate algorithms for automatic activity detection. Such algorithms would identify the activity the patient in engaged from the monitored load distribution and joint angles. Once the activity is detected, analyses can be performed and displayed/stored. Analyses may also include and overall assessment of the frequency and type of activity the patient is engaged in.

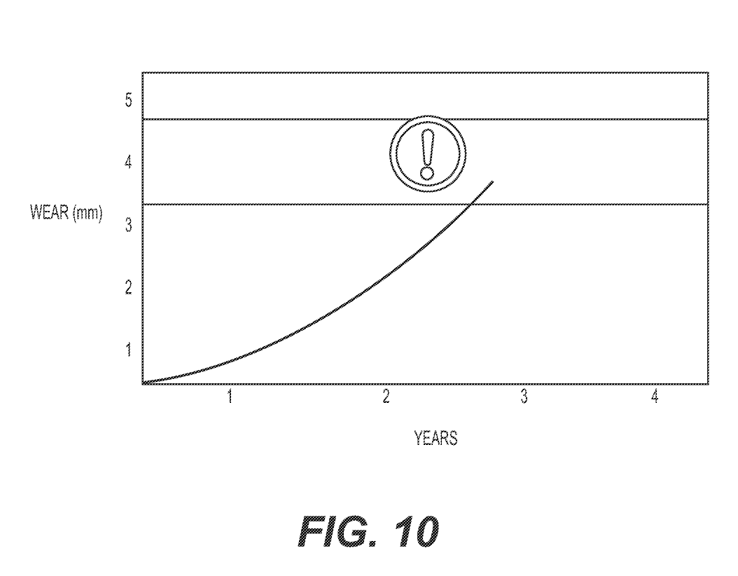

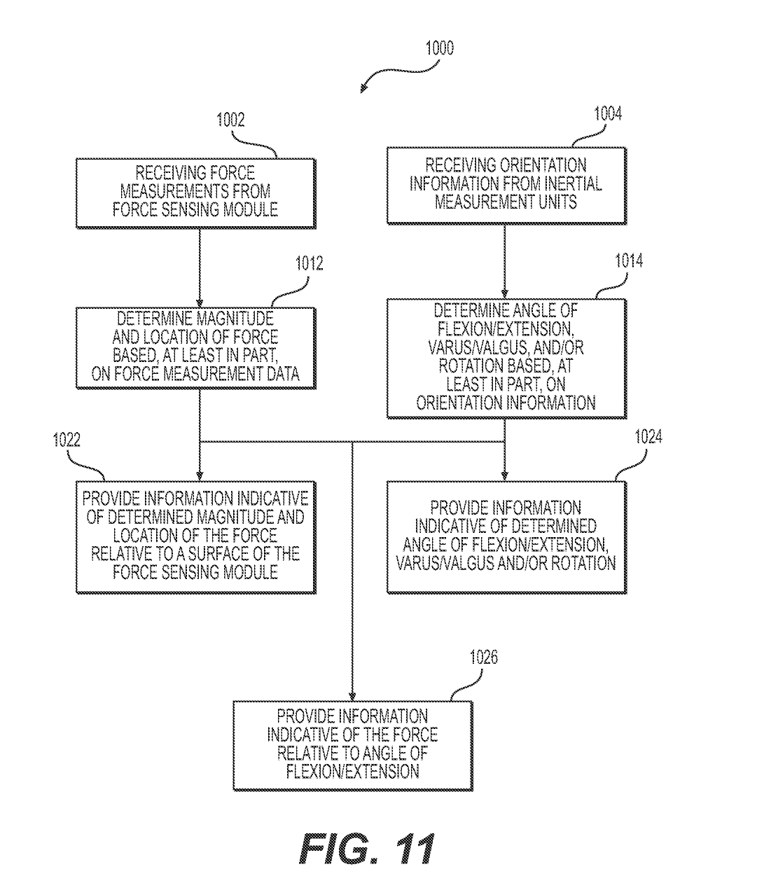

Periodic collection and trending of the load and activity information can be performed against the baseline information collected after surgery. This trend information can provide early warning of issues. FIG. 9 provides an exemplary screenshot of such a trend that displays excessive load values on the medial side during walking and warns the surgeon via a flag. Similarly, FIG. 10 provides an exemplary screenshot of another trend that displays excessive wear of the bearing surface over time and warns the surgeon via a flag.

Processing system 150 may also be configured to post-operatively aggregate results for a number of different patients. This data can be coupled with post-operative surveys in order to ascertain correlations between the post-operative kinetic and/or kinematic data (such as the WOMAC index). This type of analysis may be particularly useful in allowing surgeons to identify, using information for a variety of patients, specific load balance combinations and tolerances that result in maximum patient comfort and performance.

It will be apparent to those skilled in the art that various modifications and variations can be made to the disclosed systems and associated methods for measuring performance parameters in orthopedic prosthetic joints. Other embodiments of the present disclosure will be apparent to those skilled in the art from consideration of the specification and practice of the present disclosure. It is intended that the specification and examples be considered as exemplary only, with a true scope of the present disclosure being indicated by the following claims and their equivalents.

1. A computer-implemented method for tracking performance parameters associated with a prosthetic orthopedic articular joint, the method comprising:
receiving, at a processor associated with a computer, first information indicative of a plurality of forces detected at an articular interface between a first bone and a second bone of a patient;
receiving, at the processor, second information indicative of an orientation of at least one of the first bone and the second bone;
estimating, by the processor, a respective magnitude of each of the forces detected at the articular interface, the estimated magnitude of each of the forces based, at least in part, on the first information;
estimating, by the processor, an orientation angle associated with at least one of the first bone and the second bone relative to a reference axis, wherein the orientation angle is at least partially based on the second information; and
providing, by the processor, third information indicative of the estimated magnitude of each of the forces relative to the orientation angle associated with the at least one of the first bone and the second bone relative to the reference axis.

2. The method of claim 1, further comprising:
estimating, by the processor, a respective location of each of the forces detected at the articular interface, wherein the estimated location of each of the forces is at least partially based on the first information;
wherein the third information is further indicative of the estimated location of each of the forces detected at the articular surface.

3. (canceled)

4. (canceled)

5. (canceled)

6. (canceled)

7. The method of claim 1, wherein receiving second information indicative of the orientation of the at least one of the first bone and the second bone includes receiving information indicative of a rate of angular rotation of the at least one of the first bone and the second bone and information indicative of linear acceleration of the at least one of the first bone and the second bone, wherein estimating the orientation angle associated with the at least one of the first bone and the second bone relative to the reference axis is based, at least in part, on the information indicative of the rate of angular rotation and the information indicative of the linear acceleration.

8. A computer-implemented method for tracking performance parameters associated with a prosthetic orthopedic articular joint, the prosthetic orthopedic articular joint comprising a bearing having a bearing surface, the method comprising:
receiving, at a processor associated with a computer, first information indicative of wear of the bearing surface detected at an articular interface between a first bone and a second bone of a patient;
receiving, at the processor, second information indicative of the time between the patient receiving the prosthetic orthopedic joint and each instance of the first information;
estimating, by the processor, a rate of wear of the bearing surface for any given time period based at least in part on the first information and the second information;
estimating, by the processor, total wear of the bearing surface at any given time based at least in part on the first information and the second information.

9. The method of claim 8, further comprising displaying, on a user interface, at least one of the rate of wear and the total wear of the bearing surface.

10. (canceled)

11. (canceled)

12. An implantable sensing module for measuring performance parameters associated with a prosthetic orthopedic articular joint, comprising:
a first set of sensors disposed within a housing, the first set of sensors being mechanically coupled to a medial portion of an articular surface and configured to detect information indicative of a first force incident upon the medial portion of the articular surface;
a second set of sensors disposed within the housing, the second set of sensors being mechanically coupled to a lateral portion of the articular surface and configured to detect information indicative of a second force incident upon the lateral portion of the articular surface, and
at least one inertial measurement unit configured to detect information indicative of an orientation associated with the implantable sensing module.

13. The implantable sensing module of claim 12, further comprising a processor configured to estimate, based at least in part on the force values detected by the first set of sensors, a magnitude and a location of a force associated with the first force incident upon the medial portion of the surface, or estimate, based at least in part on the force values detected by the second set of sensors, a magnitude and a location of a center of force associated with the second force incident upon the lateral portion of the articular surface.

14. (canceled)

15. The implantable sensing module of claim 12, wherein the first set of sensors includes a transducer, the transducer comprising:
a respective cantilever component at least a portion of which is configured to deform in response to the first force incident upon the medial portion of the articular surface; and
a respective strain gauge coupled to the respective cantilever component and configured to measure the deformation in the respective cantilever component;
wherein at least a portion of each cantilever component associated with the transducer is mechanically supported at a proximal end by a base component.

16. (canceled)

17. The implantable sensing module of claim 12, further comprising a wireless transceiver configured to wirelessly transmit the information indicative of the first and second forces to a remote processing module.

18. (canceled)

19. The implantable sensing module of claim 12, wherein the at least one inertial measurement unit comprises at least one of a gyroscope, an accelerometer, or a magnetometer.

20. The implantable sensing module of claim 12, wherein the at least one inertial measurement unit comprises a gyroscope and an accelerometer.

21. An implantable sensing module for measuring performance parameters associated with a prosthetic orthopedic articular joint, comprising:
a first set of wear sensors mechanically coupled to a medial portion of a bearing surface and configured to detect information indicative of bearing surface wear on the medial portion of the articular surface; and
a second set of wear sensors mechanically coupled to a lateral portion of the bearing surface and configured to detect information indicative of bearing surface wear on the lateral portion of the articular surface, wherein the first set of wear sensors or the second set of wear sensors comprises a transducer, the transducer comprising a respective inductor coil component configured to measure the proximity of a metal component on the opposite side of the bearing surface where such measurement is indicative of the thickness of the bearing surface.

22. (canceled)

23. The implantable sensing module of claim 21, wherein the implantable sensing module comprises a processor configured to monitor the thickness of the bearing surface over time to determine the bearing surface wear on the medial portion or the lateral portion of the articular surface.

24. The implantable sensing module of claim 21, further comprising a wireless transceiver configured to wirelessly transmit the information indicative of bearing surface wear on the medial portion or the lateral portion of the articular surface to a remote processing module.

25. (canceled)

26. (canceled)

27. A joint monitoring system for tracking performance parameters associated with a prosthetic orthopedic articular joint that comprises an interface between a first bone and a second bone, the joint monitoring sensing system comprising:
a sensing module, at least a portion of which is configured for implantation within the prosthetic orthopedic articular joint, the sensing module configured to detect information indicative of at least a force at a portion of the surface of the sensing module;
an inertial measurement unit configured to detect information indicative of an orientation of at least one of a first bone and a second bone;
a processing device in communication with the sensing module and the inertial measurement unit and configured to:
estimate a location of the force relative to a surface of the articular joint, the estimated location based, at least in part, on the information indicative of the at least the force at the portion of the surface of the sensing module;
estimate an orientation angle associated with the at least one of the first bone and the second bone relative to a reference axis, the orientation angle, based, at least in part, on the information indicative of the orientation of the first bone and the second bone; and
provide information indicative of at least one of: the estimated location of the force relative to the surface of the articular interface, or the orientation angle associated with the at least one of the first bone and the second bone relative to the reference axis.

28. (canceled)

29. (canceled)

30. (canceled)

31. The joint monitoring system of claim 27, wherein the sensing module includes a plurality of transducers, each transducer including:
a respective cantilever component at least a portion of which is configured to deform in response to the force at the surface of the sensing module; and
a respective strain gauge coupled to the respective cantilever component and configured to measure the deformation in the respective cantilever component;
wherein at least a portion of each cantilever component associated with the plurality of transducers is mechanically supported at a proximal end by a central base component.

32. The joint monitoring system of claim 27, wherein the inertial measurement unit includes at least one of a gyroscope, an accelerometer, or a magnetometer.

33. The joint monitoring system of claim 27, wherein the inertial measurement unit includes a gyroscope and an accelerometer, and wherein the processing device is further configured to estimate the orientation angle based on information detected by the gyroscope and the accelerometer.

34. An implantable sensing module for measuring performance parameters associated with a prosthetic orthopedic articular joint, comprising:
a surface that engages with an articular surface of the prosthetic orthopedic articular joint;
a plurality of sensors mechanically coupled to the articular surface and configured to detect information indicative of at least one of force incident upon the surface, wear of the bearing surface, temperature in the proximity of the prosthetic orthopedic articular joint, and orientation of one of more bones; and
a processing device in communication with each of the plurality of sensors and configured to:
receive the information from one or more of the sensors;
estimate a location of the force relative to a boundary associated with the articular surface; and
estimate a magnitude of the force.

35. (canceled)

36. (canceled)

37. (canceled)

38. The implantable sensing module of claim 34, further comprising a wireless transceiver configured to wirelessly transmit the information from one of more of the sensors to a remote processing module.

39. The implantable sensing module of claim 34, further comprising at least one inertial measurement unit configured to detect information indicative of an orientation associated with the implantable sensing module.

40. The implantable sensing module of claim 34, wherein the at least one inertial measurement unit includes at least one of a gyroscope, an accelerometer, or a magnetometer.

41. The implantable sensing module of claim 34, wherein the at least one inertial measurement unit includes a gyroscope and an accelerometer.
